(19) United States
(12) Patent Application Publication
(10) Pub. No.: US 2016/0264558 A1
(43) Pub. Date: Sep. 15, 2016

(54) FLUOROGENIC PROTECTING GROUP

(71) Applicant: ISIS INNOVATION LIMITED, Oxford oxfordshire (GB)

(72) Inventors: Stuart CONWAY, Oxford (GB); Liam O'CONNOR, Oxford (GB); Ester HAMMOND, Oxford (GB)

(21) Appl. No.: 15/036,302

(22) PCT Filed: Nov. 13, 2014
(86) PCT No.: PCT/GB2014/053363

§ 371 Date: May 12, 2016

(30) Foreign Application Priority Data
Nov. 15, 2013 (GB) 1320211.4

(51) Int. Cl. C07D 413/12; A61K 31/42; C07D 311/58; A61K 49/00; C07D 215/06

(52) U.S. Cl. CPC C07D 413/12; A61K 49/0021; A61K 49/0052; C07D 215/06; C07D 311/58; A61K 31/42

(57) ABSTRACT

A fluorogenic compound comprising a biologically active component and a hypoxia-activated protecting group which is fluorogenic. The biologically active component is bound to the fluorogenic component at an active binding position such that activity of the biologically active compound is suppressed until the protecting group is released. The protecting group is cleaved under hypoxic conditions, releasing the active compound and a fluorescent compound. The compounds can therefore be used in the treatment of hypoxia-related disease and disorder, such as tumour, and enable imaging of the release of the biologically active compound.

### FIELD OF THE INVENTION

The invention relates to fluorogenic compounds which are cleavable under hypoxic conditions to release a biologically active compound and a fluorescent compound. These compounds are useful in the targeting of therapeutics to hypoxic cells, and concurrent imaging of the release of the therapeutic.

### BACKGROUND

Tumours exist and thrive in conditions of low oxygen concentration (hypoxia). Hypoxic tumour cells, however, have increased resistance to therapy and it has been found that the degree of tumour hypoxia correlates well with resistance to therapy. This presents a major barrier to the treatment of most solid tumours.

The hypoxic nature of tumour cells has been used to advantage in targeting particular therapies to tumour cells. For example, therapeutics can be adapted so as to be activated only under hypoxic conditions.

### SUMMARY OF THE INVENTION

The present inventors have found that hypoxia-activation can be used to simultaneously target and monitor release of a biologically active compound in a hypoxic environment. This is achieved by protecting the biologically active compound with a fluorogenic, hypoxia-activated protecting group. The biologically active compound is protected at an important group on the molecule so that when the protecting group is intact, the compound is inactive or has significantly reduced activity. The protecting group is cleaved under hypoxic conditions to release the active compound, e.g. a therapeutic, as well as a fluorescent compound. The fluorescent compound can be imaged on release, the technique thus providing a method of imaging release of the biologically active compound itself.

Accordingly, the present invention provides a fluorogenic compound comprising a biologically active component and a fluorogenic component, wherein the biologically active component is bound to the fluorogenic component at an active binding position, and wherein the fluorogenic component is cleavable from the biologically active component under hypoxic conditions to release a fluorescent compound and a biologically active compound.

### BRIEF DESCRIPTION OF THE FIGURES

FIG. 1 shows HPLC analysis of a compound of the invention before and after chemical reduction.

FIGS. 2(a) and (b) show the results of incubation of a compound of the invention with human cytochrome p450 in hypoxic and normoxic conditions.

FIGS. 3(a) and (b) show the results of an HPLC analysis of a compound of the invention following in-cell incubation under hypoxic and normoxic conditions.

FIGS. 4 and 5 show UV-Vis spectra of a compound of the invention, and other related compounds, demonstrating the fluorogenic nature of the compounds of the invention.

FIG. 6 shows the fluorescence spectra of a compound A comprising a fluorogenic component, and following reduction to produce compound B comprising the corresponding fluorescent component.

FIG. 7 shows the change in fluorescence spectrum over time during the course of reduction of compound A.

FIGS. 8a and 8b show the fluorescence behaviour of the compound A under enzymatic reducing conditions CYP450.

FIGS. 9a and 9b show the fluorescence behaviour of compound A following exposure to human liver microsomes under hypoxic and normoxic conditions.

FIGS. 10a and 10b to 14a and 14b show the fluorescence behaviour of compound A following exposure to a series of purified cytochrome P450 enzymes.

FIG. 15 shows the presence of compounds A and B following incubation of HepG2 cells with 1 μM compound A for 8 hours.

FIGS. 16a to 16c provide the results of FACS analysis for compound A exposed to HepG2 cells (FIG. 16a), OE21 cells (FIG. 16b) and HCT116 cells (FIG. 16c).

### DETAILED DESCRIPTION OF THE INVENTION

The fluorogenic compound described herein is a compound which is not itself fluorescent, but which is capable of releasing a fluorescent compound when cleaved under hypoxic conditions.

As used herein a biologically active compound is a compound which has a biological effect on a human or animal subject or an in vitro biological sample. For example, the biologically active compound may be a therapeutic compound which has an effect which is desirable or beneficial as part of a method of treatment of the human or animal body by therapy. The biologically active compound may alternatively be a diagnostic component. Biologically active substances are typically pharmacologically active. Biologically active substances include, but are not limited to, pharmaceutical drugs, peptides, proteins, vaccines, antibodies, nucleic acids, DNA, RNA and viruses.

The term biologically active component refers to the part of the fluorogenic compound which is derived from the biologically active compound. In the compounds of the invention, the biologically active component is protected by its attachment to the fluorogenic component and in this protected state is typically not itself active, or it has reduced activity. Rather, the cleavage of the fluorogenic protecting group component to provide the biologically active compound itself causes activation and leads to release of the active substance.

Examples of biologically active compounds suitable for adaptation in accordance with the invention are those which can be used in the treatment of a disease or disorder associated with hypoxia. For example, biologically active compounds which are useful in the treatment of tumour.

Alternatively, the fluorogenic compounds of the invention may be used to target healthy tissue which is naturally hypoxic, such as the kidney. Thus, for example, the biologically active compound may be a diagnostic component. In this aspect, the fluorogenic compounds can be used in imaging hypoxic tissue such as the kidney. The fluorogenic compounds may also be used to target therapeutic substances to the kidney in the treatment of kidney disease. The biologically active compound may therefore be one useful in the treatment of kidney disease.

The nature of the biologically active compound is not particularly limiting. For example, therapeutic substances that are toxic to cells, and which therefore benefit from targeting to tumour cells, may be used. In one aspect, the biologically active compound may be an inhibitor of enzymatic activity. For example, it may be an inhibitor of an enzyme associated with treatment of tumour, such as a kinase inhibitor, e.g. Checkpoint kinase 1, (Chk1), Aurora kinase, Ataxia telangiectasia and Rad3 related kinase (ATR kinase). The biologically active compound may alternatively be an inhibitor of the bromodomain-containing proteins found in the bromodomain and extra C-terminal domain (BET) family, or the bromodomain of cyclic AMP response element binding protein binding protein (CREBBP).

Biologically active compounds which are active inhibitors of these enzymes and bromodomains include the kinase inhibitors:

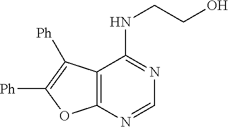 [SMILES: OCCNc1ncnc2oc(-c3ccccc3)c(-c3ccccc3)c12]

and

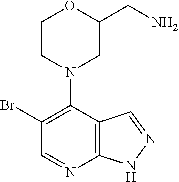 [SMILES: NCC1CN(c2c(Br)cnc3[nH]ncc23)CCO1]

and the bromodomain inhibitors:

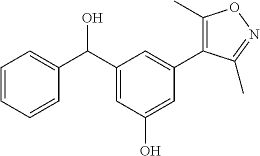 [SMILES: Cc1noc(C)c1-c1cc(O)cc(C(O)c2ccccc2)c1]

and

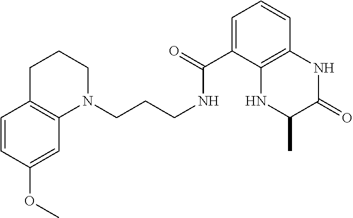 [SMILES: COc1ccc2c(c1)N(CCCNC(=O)c1cccc3c1N[C@H](C)C(=O)N3)CCC2]

Further examples are known in the art, for example angiotensin converting enzyme inhibitors or angiotensin II receptor antagonists, which may be useful in the treatment of, for example, kidney disease.

The biologically active compound is modified by attachment of a fluorogenic component at an active binding position. An active binding position is a position on the biologically active compound which is important in binding to the target, e.g. the target enzyme or bromodomain. To determine a suitable point of attachment, binding studies of the biologically active compound in question, for example X-ray crystal structures, can be used. Such studies indicate the areas on the biologically active compound which are implicated in the binding process.

Further, simple binding assays enable the skilled person to determine if a chosen position is an active position. Thus, a position can be considered an active position if the activity of the fluorogenic compound in the binding assay (i.e. a binding assay to the selected target) is reduced compared with the biologically active compound itself. Typically, activity in a binding assay will be reduced by at least 50%, preferably at least 75%, more preferably at least 90%, 95% or even 99%. Thus, the fluorogenic compound is typically substantially inactive against the target.

The binding assay can be selected based on the desired activity of the compound. Thus, for a Chk1 inhibitor biologically active compound, the fluorogenic compound has reduced activity against the biologically active compound in a Chk1-binding assay. Suitable binding assays include isothermal titration calorimetry or a cell survival assay.

The active position on the biologically active compound also acts as the leaving group on hypoxia-activated cleavage from the fluorogenic component. Thus, a functional group capable of acting as a leaving group may be selected for binding to the fluorogenic component. For example, the group may have a pKa in water of 15 or less. Examples of suitable functional groups for use in binding to the fluorogenic component include alcohols, thiols, carboxylic acids, phosphate groups, sulfate groups, sulfonamides, carbamate groups, amino groups and nitrogen atoms, e.g. as part of a ring system such as an imidazole nitrogen atom. Alcohol or thiol groups, in particular alcohol groups, are preferred.

The fluorogenic component is cleavable from the biologically active component under hypoxic conditions. Hypoxic conditions are conditions wherein the oxygen concentration is reduced compared with normal physiological conditions. Typically hypoxic conditions, or hypoxia, indicates an oxygen tension of 3% or less, but more broadly represents the conditions in which oxygen demand for normal cell viability exceeds oxygen supply.

Any appropriate mechanism of hypoxia-induced cleavage may be used. For example, the fluorogenic component may be reduced by nitroreductase enzymes to produce a fluorescent product. Cytochrome P450 reductase enzymes (CYP) are examples of such nitroreductase enzymes. The skilled person can accordingly determine whether any given compound is cleavable under hypoxic conditions by exposing the compound to CYP under hypoxia (typically oxygen tension of 3% or less) and detecting the cleaved biologically active and/or fluorescent compound. A suitable assay is described in Example 4 below.

An example of the mechanism of hypoxic cleavage is set out in Scheme 1 below:

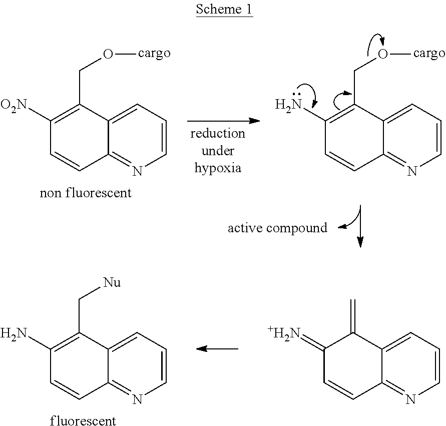

In Scheme 1, the 1,2-nitrophenyl moiety (present here fused to a pyridine ring, i.e. as a 6-nitroquinoline) is reduced under hypoxic conditions leading to release of an active compound. As depicted the biologically active component is bound via an alcohol active position (cargo-O—) to the fluorogenic component and the biologically active compound (cargo-OH) is released.

Cleavage also results in fluorescence and releases a highly active imine methide derivative which will quickly react with a nucleophile such as water or a thiol group to provide a fluorescent compound having an amine group in place of the original nitro group. Nucleophiles likely to react with the imine methide are those present in the vicinity of the compound on hypoxic release, e.g. water or a thiol such as a proximal cysteine thiol. Thus, Nu in scheme 1 above may be represented by OH, SH or S—R where R is a protein, protein fragment or peptide, typically it is OH.

A similar mechanism of action to that depicted in Scheme 1 can be achieved using a fluorogenic component having a nitroaryl, nitroheteroaryl, azidoaryl or azidoheteroaryl group, e.g. a nitroaryl or nitroheteroaryl group, linked to the bond to the biologically active component. For example, where a nitroaryl or nitroheteroaryl group is present, preferred such nitroaryl and nitroheteroaryl groups include a nitrophenyl, nitrofuryl, nitrothienyl or nitroimidazolyl group, wherein the nitro group and the bond to the biologically active component are linked to the ring on adjacent carbon atoms, or on conjugated carbon atoms, i.e. carbon atoms which are separated by an odd number of bonds around the unsaturated ring, e.g. 3 or 5 bonds. Similarly, for an azidoaryl or azidoheteroaryl group, preferred such azidoaryl and azidoheteroaryl groups include an azidophenyl, azidofuryl, azidothienyl or azidoimidazolyl group, wherein the azido group and the bond to the biologically active component are linked to the ring on adjacent carbon atoms, or on conjugated carbon atoms, i.e. carbon atoms which are separated by an odd number of bonds around the unsaturated ring, e.g. 3 or 5 bonds.

Thus, for example, for a phenyl ring, the nitro or azido group is linked in a 1,2- or 1,4-fashion relative to the bond to the biologically active component. For a furyl, thienyl or 1H-imidazolyl ring, the nitro or azido group is linked in a 2,3-, 3,2-, 2,5- or 5,2-fashion, or in the case of furyl and thienyl in a 3,4-fashion, relative to the biologically active component.

Such a structure can be schematically depicted as:

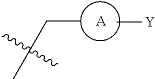

wherein the wavy line indicates the bond to the biologically active component; A comprises an aryl or heteroaryl moiety, typically a phenyl, furyl, thienyl or imidazolyl moiety; Y is —NO₂ or —N₃, e.g. —NO₂; and the group Y and the bond to the biologically active component are linked to the aryl or heteroaryl moiety, typically the phenyl, furyl, thienyl or imidazolyl moiety, on adjacent carbon atoms, or on carbon atoms which are separated by an odd number of bonds around the ring.

Preferably, A comprises a phenyl moiety and the group Y and the bond to the biologically active group are linked in a 1, 2- or 1, 4-manner relative to one another.

Thus, for example, the structure may be depicted as one of the following P, Q or S, in particular P:

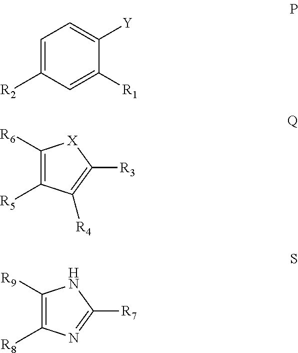

wherein in formula P, one of R₁ and R₂ represents —CH₂—B₁; and Y represents —NO₂ or —N₃, e.g. —NO₂;
in formula Q, X represents NH or O; and either (a) R₃ and R₄ independently represent Y and —CH₂—B₁; or (b) R₄ and R₅ independently represent Y and —CH₂—B₁; or (c) R₃ and R₅ independently represent Y and —CH₂—B₁; where Y represents —NO₂ or —N₃, e.g. —NO₂; and in formula S, either (a) R₈ and R₉ independently represent Y and —CH₂—B₁; or (b) R₉ and R₇ independently represent —Y and —CH₂—B₁; where Y represents —NO₂ or —N₃, e.g. —NO₂; where B₁ is the biologically active component;
and wherein any ring carbon or nitrogen atom which is not bound to —Y or —CH₂—B₁ may be unsubstituted, carry a further substituent or be linked to a further aryl or heterocyclic ring.

In one embodiment, in formulae P, Q and S, Y is —NO₂.

The fluorogenic component is not itself fluorescent (i.e. it is non-fluorescent), but releases a fluorescent compound on cleavage. In this context, non-fluorescent implies that, at the emission wavelength of the fluorescent compound released, the fluorogenic compound will typically have at least 50% reduced emission intensity, preferably at least 75% or 90% reduced emission intensity, compared with the fluorescent compound released.

In the example above, therefore, A is non-florescent when bound to the nitro or azido group, but fluorescent when the nitro or azido group is replaced with an amine group. Put another way, A is selected such that -A-Y is non-fluorescent but -A-NH₂ is fluorescent. Thus, the resulting compound having the structure:

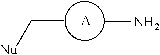

where Nu is a nucleophilic moiety as described above, is the fluorescent compound released. Typically, in the fluorescent compound, Nu is OH.

In one aspect, known fluorescent compounds having an amine-substituted phenyl, furyl, thienyl or imidazolyl group or moiety can be adapted for use in the invention. An example is 6-aminoquinoline. Such compounds can be adapted by replacing the amine group with a nitro group or an azido group (e.g. a nitro group), and by appropriate linkage to a biologically active component, to provide a fluorogenic compound in accordance with the invention. 6-Aminoquinoline can be adapted as depicted in Scheme 1. In this case, A is quinoline and the nitro group and the bond to the biologically active component are linked to the phenyl moiety of the quinoline group in a 1,2- or 1,4-fashion. A particular example of a fluorogenic component based on quinoline is:

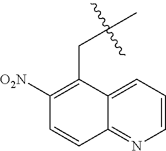 [SMILES: *Cc1c([N+](=O)[O-])ccc2ncccc12]

A further example is:

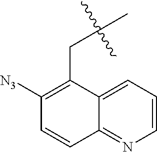 [SMILES: *Cc1c(N=[N+]=[N-])ccc2ncccc12]

Alternatively, the phenyl, furyl, thienyl or imidazolyl group can be fused or conjugated to a known fluorophore, e.g. the fluorogenic component may be of formula F-L-B(Y)—CH₂—, where Y is —NO₂ or —N₃, e.g. —NO₂; F is a fluorophore which fluoresces when the NO₂ or N₃ group is replaced with NH₂; B is a phenyl, furyl, thienyl or imidazolyl ring wherein the group Y and the bond to the biologically active component are linked to B on adjacent carbon atoms, or on carbon atoms which are separated by an odd number of bonds around the ring (i.e. they are conjugated); and L is a linker which conjugates the fluorophore to B, e.g. L may be a bond or an ethenylene group: F—B(Y)—CH₂— or F—CH═CH—B(Y)—CH₂—. Examples are F—B(NO₂)—CH₂—, F—CH═CH—B(NO₂)—CH₂—, F—B(N₃)—CH₂— and F—CH═CH—B(N₃)—CH₂—. In one embodiment, Y is NO₂, so the group is of formula F-L-B(NO₂)—CH₂—.

An example is a compound of formula:

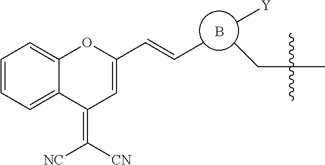

wherein the wavy line indicates the bond to the biologically active component; Y is —NO₂ or —N₃, e.g. —NO₂; and B is a phenyl, furyl, thienyl or imidazolyl ring wherein the nitro or azido group and the bond to the biologically active component are linked to B on adjacent carbon atoms, or on carbon atoms which are separated by an odd number of bonds around the ring.

Preferably, B is phenyl and the nitro or azido group and the bond to the biologically active group are linked to the phenyl ring in a 1, 2- or 1, 4-manner relative to one another.

A specific example is the compound:

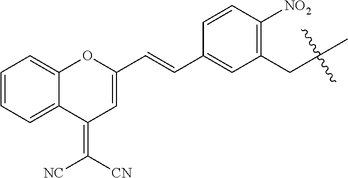 [SMILES: *Cc1cc(/C=C/C2=CC(=C(C#N)C#N)c3ccccc3O2)ccc1[N+](=O)[O-]]

Another specific example is the compound:

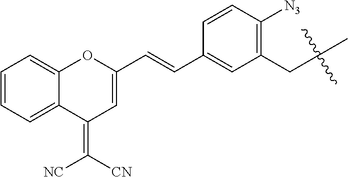 [SMILES: *Cc1cc(/C=C/C2=CC(=C(C#N)C#N)c3ccccc3O2)ccc1N=[N+]=[N-]]

Typically, and as is the case in the examples above, the phenyl, furyl, thienyl or imidazolyl group of A (or of P, Q or S) is further linked to one or more, e.g. one, two or three, typically one or two, for example one, further aryl or heterocyclyl moieties in order to provide a fluorescent compound on release. The phenyl, furyl, thienyl or imidazolyl group may be linked in a fused or conjugated fashion to the one or more further aryl or heterocyclyl moieties. Where rings are conjugated this may be via a direct bond or, in the case of a phenyl ring, via a C₂-C₄ alkenyl or C₂-C₄ alkynyl group, e.g. ethenyl or ethynyl, in particular ethenyl.

An aryl moiety may be, for example, a further phenyl ring or two or more (e.g. 2, 3 or 4) fused phenyl rings such as naphthyl. A heterocyclic or heterocyclyl moiety may be, for example, a 5- to 12-membered, e.g. a 5 or 6-membered ring containing one or more, e.g. 1, 2 or 3, heteroatoms selected from N, O and S. Such heterocyclyl rings are typically unsaturated or partially unsaturated. Examples of heterocyclyl rings include pyridyl, pridazinyl, pyrimidinyl, pyrazinyl, pyrrolyl, pyrazolyl, imidazolyl, triazolyl, furyl, oxazolyl, isoxazolyl, thienyl, thiazolyl, isothiazolyl and triazinyl, and fused rings including indolyl, isoindolyl, imidazolyl, indazolyl, benzofuranyl, benzothiophenyl, benzoisothiazolyl, benzooxazolyl, benzoisoxazolyl and chromenyl.

A (or in its preferred form P, Q or S) carries at least the group Y (e.g. nitro) and a bond to the biologically active component and may carry one or more, e.g. 1, 2 or 3, further substituent groups R which may be the same or different. Suitable substituents R include C₁₋₆ alkyl, C₂₋₆ alkenyl, C₂₋₆ alkynyl, amine, C₁₋₄ alkylamine, di(C₁₋₄ alkyl)amine, cyano, sulphate and phosphate, where any alkyl, alkenyl or alkynyl substituent may be further substituted with one or more e.g. 1 or 2, amine, cyano or unsubstituted C₁₋₄alkylamine or di(C₁₋₄ alkyl)amine groups. Substituents linked to the ring by a double bond, e.g. imine (C═NR), methylene (C═CRR) and carbonyl (C═O) are also envisaged, where each R is the same or different and is as defined above.

Where multiple aryl and/or heterocyclyl groups are present, these may form one or more fused structures. For example, 1, 2, 3 or 4 rings may be fused to provide a fused ring structure.

The fluorogenic component releases a fluorescent compound on hypoxia-induced cleavage such that imaging of the released compound can be carried out. The florescent compound typically fluoresces at UV and/or UV/visible wavelengths. Suitable wavelengths of emissions are, for example from 300 to 800 nm, e.g. from 400 to 500 nm.

The compounds described herein can be synthesised by routes known in the art. The biologically active component can be attached to the fluorogenic component by any means available to the skilled chemist. For example, a fluorogenic component containing a leaving group at the position which is to connect to the biologically active component can be reacted with a functional group on the biologically active component. An example of the synthesis of a fluorogenic compound according to the invention is described further below.

The fluorogenic compounds may be in the form of pharmaceutically acceptable salts. As used herein, a pharmaceutically acceptable salt is a salt with a pharmaceutically acceptable acid or base. Pharmaceutically acceptable acids include both inorganic acids such as hydrochloric, sulfuric, phosphoric, diphosphoric, hydrobromic or nitric acid and organic acids such as citric, fumaric, maleic, malic, ascorbic, succinic, tartaric, benzoic, acetic, methanesulfonic, ethanesulfonic, benzenesulfonic or p-toluenesulfonic acid. Pharmaceutically acceptable bases include alkali metal (e.g. sodium or potassium) and alkali earth metal (e.g. calcium or magnesium) hydroxides and organic bases such as alkyl amines, aralkyl amines and heterocyclic amines.

The fluorogenic compounds may also be provided as a pharmaceutical composition comprising a fluorogenic compound as defined above and a pharmaceutically acceptable carrier or diluent.

Said pharmaceutical composition typically contains up to 85 wt % of a compound of the invention. More typically, it contains up to 50 wt % of a compound of the invention. Preferred pharmaceutical compositions are sterile and pyrogen free.

The fluorogenic compounds of the invention are useful in the treatment of diseases or disorders relating to hypoxia or in which hypoxic cells need to be targeted by therapeutic compounds. A hypoxia-related disease or disorder as used herein refers to any disease or disorder which is treatable with therapeutic agents which are targeted to hypoxic cells. Thus, such a disease or disorder is typically one which presents, or predominantly presents, in hypoxic cells in the body. Thus, those diseases and disorders which are known to be treatable with a hypoxia-activated therapeutic compound, can also be treated with the compounds of the invention.

An example of a hypoxia-related disease or disorder is tumour. Accordingly, the compounds of the invention are useful in the treatment of tumour. Typically, treatment of tumour will be carried out using a fluorogenic compound having a biologically active component known to be useful in the treatment of tumour. For example, the biologically active component may be derived from a Chk1 kinase inhibitor, aurora kinase inhibitor or a bromodomain inhibitor as described above.

A further example of a hypoxia-related disease or disorder is a disease or disorder of a hypoxic organ, e.g. the kidney. The fluorogenic compounds of the invention are therefore useful in the treatment of kidney diseases or disorders.

The fluorogenic compounds of the invention are also useful in the treatment of bacterial infection on the cystic fibrosis lung and bacterial biofilms.

The invention therefore provides a fluorogenic compound or composition as described herein for use in a method of treatment of a hypoxia-related disease or disorder, in particular tumour. Also provided is a method of treatment a hypoxia-related disease or disorder, in particular tumour, in a human or animal subject comprising administering to the subject an effective amount of a fluorogenic compound or composition as described herein. Also provided is the use of a fluorogenic compound or composition as described herein in the manufacture of a medicament for the treatment of a hypoxia-related disease or disorder, in particular tumour. The disease or disorder may be a disease or disorder of a hypoxic organ, e.g. the kidney.

A particular benefit of the fluorogenic compounds of the invention is the ability to image the release of the biologically active compound by use of fluorescence imaging techniques. Thus, the invention also provides the use of the fluorogenic compounds of the invention in a method of fluorescence imaging. Also provided is a method of carrying out fluorescence imaging on a human or animal subject which comprises administering an effective amount of a fluorogenic compound of the invention to the subject and carrying out fluorescence imaging. The method is typically carried out as part of a method of therapy, in particular a therapy of a hypoxia-related disease or disorder.

The nature of the fluorescence imaging technique used is not particularly limited. Typically, the compounds described herein release fluorescent compounds which emit in the UV or UV/visible wavelengths and accordingly an imaging technique suitable for the particular wavelength is selected.

The imaging may be carried out simultaneously with administration of the fluorogenic compound or imaging may begin after administration. Typically, imaging is begun 5 minutes, 10 minutes, 20 minutes or for example 30 minutes after administration. The time lapse between administration and imaging will depend on the route of administration selected and the nature of the biologically active compound. Imaging may be carried out over a period of time to monitor release of the biologically active compound.

The fluorogenic compounds of the invention may be administered by any suitable route, depending on the nature of the method of treatment, e.g. orally (as syrups, tablets, capsules, lozenges, controlled-release preparations, fast-dissolving preparations, etc); topically (as creams, ointments, lotions, nasal sprays or aerosols, etc); by injection (subcutaneous, intradermic, intramuscular, intravenous, etc.), transdermally (e.g. by application of a patch, gel or implant) or by inhalation (as a dry powder, a solution, a dispersion, etc).

The compounds of the invention are typically formulated for administration with a pharmaceutically acceptable carrier or diluent. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non toxic and pharmacologically inactive substances used in pharmaceutical formulations. Such pharmaceutical preparations may be manufactured in known manner, for example, by means of mixing, granulating, tableting, sugar coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspension or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for injection or infusion may contain as carrier, for example, sterile water or preferably they may be in the form of sterile, aqueous, isotonic saline solutions.

An amount of fluorogenic compound to be administered as part of a method of treatment will depend on, for example, the identity of the biologically active component and can be determined by one of skill in the art. Thus, the dose of the fluorogenic compound will typically be equivalent to or less than the dose of the biologically active component present if administered alone, i.e. the amount of biologically active component present in the compound administered will typically be the same or less than the amount that would be administered if in free form.

The following Examples illustrate the invention. They do not however, limit the invention in any way.

### EXAMPLES

### General Experimental

¹H NMR spectra were recordered on Bruker DPX400 (400 MHz), Bruker DQX400 (400 MHz) and Bruker AV500 (500 MHz) spectrometers using deterochloroform (unless otherwise indicated) as a reference for the internal deuterium lock. The chemical shift data for each signal are given as δH in units of parts per million (ppm) relative to tetramethylsilane (TMS) where δ (TMS)=0.00 ppm. The multiplicity of each signal is indicated by: s (singlet); d (doublet); t (triplet); q (quartet); qn (quintet); dd (doublet of doublets); ddd (double of doublet of doublets), m (multiplet) or br s (broad singlet). The number of protons (n) for a given resonance signal is indicated by nH. Coupling constants (J) are quoted in Hz and are recorded to the nearest 0.1 Hz. Identical proton coupling constants (J) are averaged in each spectrum and reported to the nearest 0.1 Hz. The coupling constants are determined by analysis using Bruker TopSpin software.

¹³C NMR spectra were recordered on Bruker AV500 (125.8 MHz) and Bruker (100.6 MHz) spectrometers using a DEPT pulse sequence with broadband proton decoupling and internal deuterium lock. The chemical shift data for each signal are given as δC in units of parts per million (ppm) relative to tetramethylsilane (TMS) where δC (TMS)=0.00 ppm. Where appropriate, coupling constants (J) are quoted in Hz and are recorded to the nearest 0.1 Hz. ¹H and ¹³C spectra were assigned using 2D NMR experiments including COSY, HMBC and HSQC.

¹⁹F NMR spectra were recorded on a Bruker AV400 (376.6 MHz) spectrometer using a broadband proton decoupling pulse sequence and deuterium internal lock. The chemical shift for each signal are given as δF in units of parts per million (ppm) relative to trifluoromethane where δF=0.00 ppm.

Mass Spectra were acquired on either a Micromass LCT Premier Spectrometer (low resolution) or Bruker MicroTOF spectrometer (high resolution) using electrospray ionization, operating in positive or negative mode, from solutions of acetonitrile or methanol. m/z values are reported in Daltons and followed by their percentage abundance in parentheses.

Melting Points were determined using a Leica Galen III hot stage microscope and are uncorrected.

Infrared Spectra were obtained from neat samples, either as solids or liquids, using a diamond ATR module. The spectra were recorded on a Bruker Tensor 27 spectrometer. Absorption maxima are reported in wavenumbers (cm⁻¹) and reported as s (strong), m (medium) or w (weak).

Analytical thin layer chromatography (TLC) was carried out on normal phase Merck silica gel 60 F254 aluminium-supported thin layer chromatography sheets, unless otherwise stated. Visualisation was by absorption of UV light (λₘₐₓ 254 or 365 nm), exposure to iodine vapour or thermal development after dipping in one of: a ethanolic solution of: phosphomolybdic acid (PMA), ninhydrin, vanillin and sulphuric acid; b aqueous solution of potassium permanganate.

Flash column chromatography was performed on a Biotage SP1 System using KP-Sil™ cartridges or carried out manually on VWR Prolabo silica gel 60 (240-400 mesh), eluting with solvents as supplied, under a positive pressure of compressed air (unless otherwise stated). Reverse phase column chromatography was carried out on Fluka Ltd silica gel 100 C18-reversed phase under a positive pressure of compressed nitrogen.

Anhydrous solvents were obtained under the following conditions: Diethyl ether, toluene, dichloromethane and tetrahydrofuran were dried by passing them through a column of activated basic alumina according to the Grubbs procedure.²⁶ Anhydrous N,N-dimethylformamide and methanol were purchased from SigmaAldrich UK in SureSeal™ bottles and used without further purification. All other solvents were used as supplied (analytical or HPLC grade) without purification.

Chemicals were purchased from Acros UK, Apollo Scientific, Enamine, Sigma Aldrich UK, Alfa Aesar UK, Fisher UK, Fluka UK, Fluorochem, Merck, Argo International Limited or TCI-Europe. All solvents and reagents were purified, when necessary, by standard techniques. Where appropriate and if not otherwise stated, all non-aqueous reactions were performed in a flame-dried flask under an inert atmosphere.

In vacuo refers to the removal of solvent under reduced pressure using a Buchi™ rotary evaporator.

Brine refers to a saturated solution of sodium chloride.

Petroleum Ether refers to the fraction of light petroleum ether boiling in the range 40-60° C., unless otherwise stated.

Lyophilizer removal of water from aqueous solution by freeze drying using a Christ Alpha 2-4-LD lyophilizer.

Microanalyses were obtained from the Elemental Analysis Service, London Metropolitan University, London.

cLogP values were calculated using Advanced Chemistry Development (ACD) Laboratories' ACD/ChemSketch Freeware software, version 12.0.

### Example 1a

### Synthesis of Fluorogenic Component

### 5-Bromo-6-nitroquinoline (15)

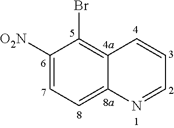 [SMILES: O=[N+]([O-])c1ccc2ncccc2c1Br]

6-nitroquinolin-5-amine (5 g, 25.5 mmol, 1 eq.) and CuBr (4.55 g, 31.8 mmol, 1.2 eq) were added to a stirred solution of H₂O (250 mL) and conc. aq. HBr (48% w/v, 125 mL). An aq. solution of NaNO₂ (7.9 M, 79 mmol, 3 eq.) was added dropwise to the resulting mixture, and left to stir for 3 h. The reaction was neutralised to pH 7 with sat. aq. NaHCO₃, sat. EDTA solution (15 mL) added and the mixture left to stir for 30 min. The product was extracted with EtOAc (5×50 mL) and the combined organic layers were washed with sat. EDTA solution (3×30 mL), saturated Na₂S₂O₃ solution (3×30 mL), brine (3×30 mL) and concentrated in vacuo. The resulting residue was adsorbed onto Celite® and purified using silica gel chromatography, eluting with EtOAc/PE (1:2), yielding 15 (3.41 g, 13.5 mmol, 51%) as a colourless solid: R_{f}: 0.41 (EtOAc:PE 2:3); mp: 138-140° C. (EtOAc)

### 6-Nitroquinoline-5-carbaldehyde (16)

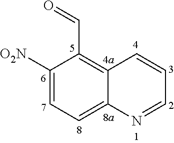 [SMILES: O=Cc1c([N+](=O)[O-])ccc2ncccc12]

Compound 15 (50 mg, 0.20 mmol, 1 eq.) in distilled THF (2 mL) was cooled to −78° C. Phenyl Lithium (1.75 M soln. in THF) (113 μL, 0.2 mmol, 1 eq) was added to the solution dropwise over 10 min, and the reaction left to stir for 30 min at −78° C. Distilled ethyl formate (160 μL, 1.0 mmol, 5 eq.) was added dropwise over 15 min at −78° C., the reaction left to gradually warm to rt, and stirred for 18 h. The reaction mixture was then concentrated in vacuo, the resulting residue adsorbed onto Celite® and purified using silica gel chromatography, eluting with EtOAc/PE (2:3) followed by recrystallisation from MeCN, yielding 16 (18 mg, 0.089 mmol, 45%) as pale orange needles: R_{f}: 0.19 (EtOAc:PE 1:1); mp: 165-167° C. (MeCN).

### (6-Nitroquinolin-5-yl)methanol (8)

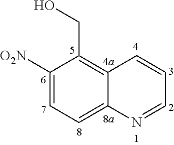 [SMILES: O=[N+]([O-])c1ccc2ncccc2c1CO]

A solution of 16 (100 mg, 0.50 mmol, 1 eq.) in distilled THF (4 mL) was cooled to 0° C. NaBH₄ (9 mg, 0.50 mmol, 1 eq.) was added portionwise and the reaction left to stir for 15 min at 0° C. MeOH (2 mL) and aq. HCl (1 mL, 1 M) were added and the reaction mixture was concentrated in vacuo. The resulting residue was dissolved in EtOAc (15 mL) and washed with H₂O (3×15 mL), saturated NaHCO₃ (2×10 mL), dried over MgSO₄, and concentrated in vacuo. The resulting residue was adsorbed onto Celite®, and purified using silica gel chromatography, eluting with EtOAc/PE (3:2), yielding 8 (95 mg, 0.47 mmol, 94%) as a pale yellow solid: R_{f}: 0.30 (EtOAc); mp: 139-141° C. (EtOAc); νₘₐₓ (solid) cm⁻¹: 1324 (NO₂ sym, s), 1532 (NO₂ asym, s), 1601 (C═C Ar, w), 3102 (OH, b); ¹H NMR (500 MHz, CDCl₃): 5.14 (s, 2H, CH₂OH), 7.65 (dd, 1H, J, =8.7, J₂=4.2, C³H), 8.11 (d, 1H, J=9.2, C⁷H), 8.23 (d, 1H, J=9.2, C⁸H), 8.79 (d, 1H, J=8.7, C⁴H), 9.09 (dd, 1H, J₁=4.2, J₂=1.6, C²H); ¹³C NMR (125 MHz, CDCl₃): 55.7 (C^{5′}), 123.2 (C³), 123.7 (C⁷), 127.5 (C^{4a}), 131.9 (C⁸), 132.5 (C⁵), 134.6 (C⁴), 147.8 (C⁶), 149.6 (C^{8a}), 153.2 (C²); HRMS: m/z (EI/FI)⁺[Found (M)⁺ 204.0541, (M[D])⁺ 205.0533. C₁₀H₈N₂O₃ requires (M)⁺ 204.0535, (M[D])⁺ 205.0565]; m/z (ES⁺) 205.1 ([M+H]⁺, 100%); Anal. calculated for: C, 58.8; H, 4.0; N, 13.7. Found: C, 58.8; H, 3.8; 13.7.

### 5-(Bromomethyl)-6-nitroquinoline (17)

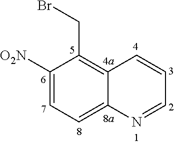 [SMILES: O=[N+]([O-])c1ccc2ncccc2c1CBr]

A solution of 8 (20 mg, 0.10 mmol, 1 eq.) in conc. aq. HBr (48 w/v, 0.2 mL) was stirred at 75° C. for 20 h. The reaction was allowed to cool to rt, neutralised to pH 7 using solid K₂CO₃. The mixture was extracted with EtOAc (3×3 mL) and the combined organic layers dried over MgSO₄, and concentrated in vacuo yielding 17 (22 mg, 0.08 mmol, 84%) as orange crystals: R_{f}: 0.33 (EtOAc:PE 1:1); mp: 112-114° C. (EtOAc); νₘₐₓ (solid) cm⁻¹: 3089/3050 (ArCH, w), 1518 (NO₂, asym, s), 1343 (NO₂, sym, s); ¹H NMR (500 MHz, CDCl₃): δ 5.11 (s, 2H, CH₂Br), 7.70 (dd, 1H, J₁=8.7, J₂=4.2, C³H), 8.14 (d, 1H, J=9.2, C⁷H), 8.24 (s, 1H, J=9.2, C⁸H), 8.67 (d, 1H, J=8.7, C⁴H), 9.11 (dd, 1H, J₁=4.2, J₂=1.6, C²H); ¹³C NMR (125 MHz, CDCl₃): δ 22.6 (CH₂Br), 123.1 (C³), 124.4 (C⁷), 126.6 (C^{4a}), 129.3 (C⁵), 132.6 (C⁸), 133.9 (C⁴), 146.6 (C⁶), 149.5 (C^{8a}), 153.3 (C²); HRMS: m/z (EI/FI)⁺ [Found (M[⁷⁹Br])⁺ 265.9686, (M[⁸¹Br])⁺ 267.9675. C₁₀H₇BrN₂O₂ requires (M[⁷⁹Br])⁺ 265.9691, (M[⁸¹Br])⁺ 267.9670; m/z (EI⁺) 283.2 ([M+H]⁺, 100%); Anal. calculated for: C, 45.0; H, 2.6; N, 10.5. Found: C, 45.1; H, 2.8; N, 10.5.

### Example 1b

### Synthesis of Fluorogenic Compound

Active compound 3 was protected by the fluorogenic protecting group prepared in Example 1a

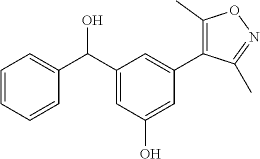 [SMILES: Cc1noc(C)c1-c1cc(O)cc(C(O)c2ccccc2)c1]

### Compound 3

### (3-(3,5-Dimethylisoxazol-4-yl)-5-((6-nitroquinolin-5-yl)methoxy)phenyl)(phenyl) methanol (18)

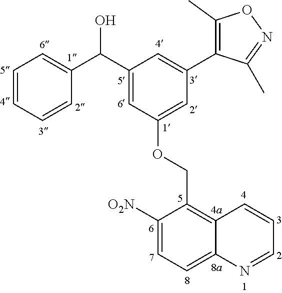 [SMILES: Cc1noc(C)c1-c1cc(OCc2c([N+](=O)[O-])ccc3ncccc23)cc(C(O)c2ccccc2)c1]

A mixture of 3 (70 mg, 0.24 mmol), Cs₂CO₃ (77 mg, 0.24 mmol, 1 eq) and DMF (1.2 mL) were stirred at rt for 20 min. A solution of 17 (63 mg, 0.24 mmol, 1 eq.) in DMF (1.2 mL) was added dropwise at rt over 5 min. The reaction mixture was left to stir for 2 h, H₂O (5 mL) was added, and the product extracted with EtOAc (2×10 mL). The aq. layer was neutralised to pH 7 with 1 M HCl, and extracted with EtOAc (2×10 mL). The combined organic layers were washed with H₂O (3×5 mL), brine (3×5 mL), dried over Na₂SO₄ and concentrated in vacuo. The resulting residue was adsorbed onto Celite®, and purified using silica gel chromatography, eluting with EtOAc/PE (50% EtOAc), yielding 18 (62 mg, 0.13 mmol, 54%) as an off white solid: R_{f}: 0.18 (EtOAc:PE 1:1); mp: 75-77° C. (Et₂O); νₘₐₓ (solid) cm⁻¹: 3324 (ArOH, b), 1531 (NO₂, asym, s), 1322 (NO₂, sym, s), 1023 (CH₂OCH₂, s); ¹H NMR (500 MHz, CDCl₃): 2.20 (s, 3H, CH₃CN/CH₃CO), 2.35 (s, 3H, CH₃CN/CH-₃CO), 5.66 (s, 2H, CH₂O), 5.86 (s, 1H, CHOH), 6.74 (s, 1H, C^{2′}H), 6.89 (s, 1H, C^{4′}H), 7.09 (s, 1H, C^{6′}H), 7.28-7.33 (m, 1H, C^{4″}H), 7.34-7.42 (m, 4H, C^{2″}H, C^{3″}H, C^{5″}H, C^{6″}H), 7.60 (dd, 1H, J₁=8.4, J₂=4.2, C³H), 8.08 (d, 2H, J=9.2, C⁷H), 8.27 (d, 2H, J=9.2, C⁸H), 8.63 (d, 1H, J=8.4, C⁴H), 9.08 (dd, 1H, J₁=4.2, J₂=1.6, C²H); ¹³C NMR (125 MHz, CDCl₃): 11.0 (CH₃CN/CH₃CO), 11.8 (CH₃CO/CH₃CN), 62.3 (CH₂O), 76.3 (CHOH), 112.0 (C^{6′}), 115.2 (C^{2′}), 116.3 (CC(CH₃)), 121.1 (C^{4′}), 123.2 (C³), 123.8 (C⁷), 126.7 (C^{2″}/C^{6″}), 127.6 (C^{4a}/C⁵), 128.2 (C^{4″}), 128.9 (C^{3″}/C^{5″}), 132.4 (C^{3′}), 132.7 (C⁸), 134.7 (C⁴), 143.5 (C^{5′}), 146.5 (C^{1′}), 148.4 (C⁶), 149.2 (C^{8a}), 153.1 (C²), 158.6 (CH₃CO/CH₃CN), 158.6 (C^{1″}), 165.6 (CH₃CO/CH₃CN); HRMS: m/z (ESI⁺) [Found (M+Na)⁺ 504.1531. C₂₈H₂₃N₃O₅ requires (M+Na)⁺ 504.1530]; LRMS: m/z (ES⁺) 482.1 ([M+H]⁺, 100%); Analytical HPLC (RPB C18 column [100 mm×3.2 mm, 35° C.]; 70:30:0.1 H2O:MeCN:TFA→95:5:0.1 MeCN:H2O:TFA; 6 min) Ret. Time=5.89 min, Purity: 96.9%.

Compound 3 is reported to have an IC₅₀ value of 0.382 μM against BRD4, as determined by an AlphaScreen assay (J Med Chem 2013, 56, 3217-3227 and Philpott et al, Mol Biosyst 2011, DOI: 10.1039/c1mb05099k). In order to determine the IC₅₀ value of compound 18 against BRD4, the same AlphaScreen assay was employed and found to have an IC₅₀ value of 0.749 μM, a 2-fold decrease in potency.

### Example 2

### Synthesis of Fluorogenic Compound

### N-(4-((6-Nitroquinolin-5-yl)methoxy)phenyl)acetamide (21)

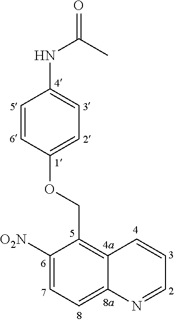 [SMILES: CC(=O)Nc1ccc(OCc2c([N+](=O)[O-])ccc3ncccc23)cc1]

A mixture of paracetamol (4-acetamidophenol) (32 mg, 0.20 mmol, 1 eq.) and Cs₂CO₃ (62 mg, 0.20 mmol, 1 eq.) in DMF (1.2 mL) was stirred at rt for 30 min. A solution of 17 (see Example 1; 50 mg, 0.20 mmol, 1 eq) in DMF (1.2 mL) was added dropwise at rt over 5 min. The reaction mixture was left to stir for 2 h, H₂O (5 mL) was added, and the product extracted with EtOAc (2×10 mL). The aq. layer was neutralised to pH 7 with 1 M aq. HCl, and extracted with EtOAc (2×10 mL). The combined organic layers were washed with H₂O (3×5 mL), brine (3×5 mL), dried over Na₂SO₄, filtered and concentrated in vacuo. The resulting residue was adsorbed onto Celite® and purified using silica gel chromatography, eluting with EtOAc/PE (9:1), to yield 21 (16 mg, 0.05 mmol, 25%) as a colourless solid: R_{f}: 0.43 (EtOAc); mp: 204-206° C. (EtOAc); νₘₐₓ (solid) cm⁻¹: 1366 (NO₂, asym, s), 1500 (Ar C—H, s), 1524 (NO₂, sym, s), 1600 (Ar C—H, m), 1650 (CONH, m); ¹H NMR (500 MHz, CDCl₃): 2.17 (s, 3H, CH₃CO), 5.62 (s, 2H, CH₂O), 6.94-6.97 (m, 2H, C^{2′}/C^{6′}), 7.09 (s, 1H, NH), 7.41-7.45 (m, 2H, C^{3′}/C^{5′}), 7.61 (dd, 1H, J₁=8.7, J₂=4.2, C³H), 8.08 (d, 1H, J=9.2, C⁷H), 8.27 (d, 1H, J=9.2, C⁸H), 8.62 (d, 1H, J=8.7, C⁴H), 9.08 (dd, 1H, J₁=4.3, J₂=1.5, C²H); ¹³C NMR (500 MHz, CDCl₃): 24.5 (COCK), 62.4 (CH₂O), 115.4 (C^{2′}, C^{6′}), 121.9 (C^{3′}, C^{5′}), 123.0 (C³), 123.8 (C⁷), 127.5 (C^{4a}), 127.7 (C⁵), 132.1 (C^{4′}), 132.3 (C⁸), 134.8 (C⁴), 148.2 (C⁶), 148.9 (C^{8a}), 152.8 (C²), 154.9 (C^{1′}), 168.5 (CO); HRMS: m/z (ESI⁺) [Found (M+Na)⁺ 360.0953. C₁₈H₁₅N₃O₄ requires (M+H)⁺ 360.0955]; m/z (ES⁺) 338.1 ([M+H]⁺, 100%); Analytical HPLC (RPB C18 column [100 mm×3.2 mm, 35° C.]; 90:10:0.1 H2O:MeCN:TFA→95:5:0.1 MeCN:H2O:TFA; 6 min) Ret. Time=5.94 min, Purity: >99%.

### Example 3

### Fragmentation on Chemical Reduction

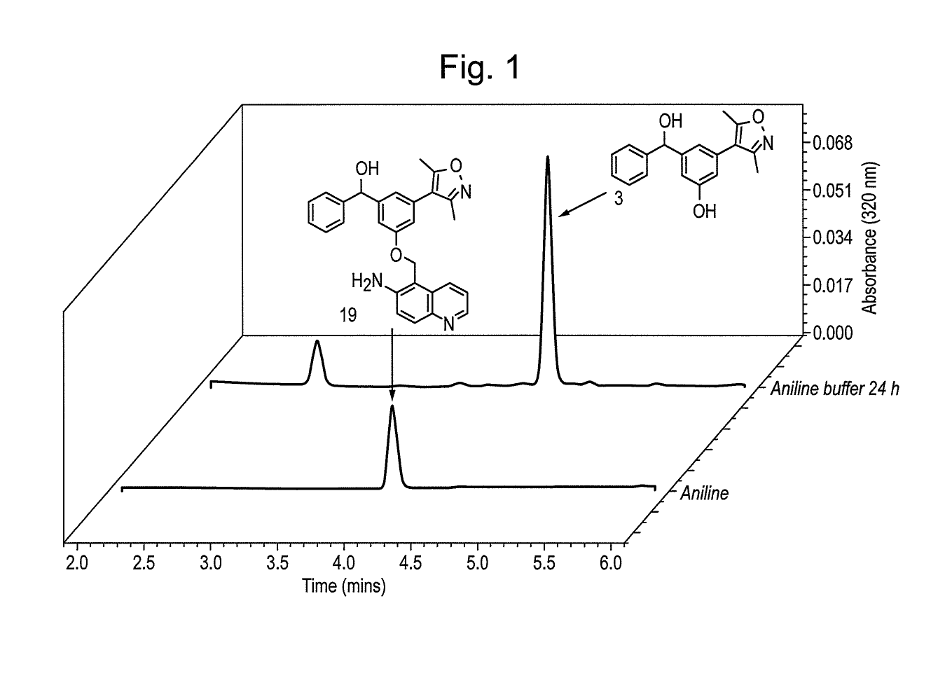

Compound 18 (Example 1) was subjected to zinc mediated nitro reduction to deliver the corresponding amine 19 (See FIG. 1), a sample of which was then taken for incubation in 0.5M KH₂ PO₄ buffer at 37° C. over 24 h.

To a solution of 19 (1 mg, 0.0021 mmol) in DMF (2 mL) was added aqueous ammonium chloride (20 μL, 10% w/v) and zinc powder (4 mg, 0.0612 mmol, 30 equiv). The resulting mixture was stirred at ambient temperature for 16 h. Aliquots (200 μL) were taken at designated times (where T=0 refers to before the addition of zinc powder), and the mixture was analysed by HPLC.

For every time point of interest, 5 μL of the T=1 h aliquot from the zinc reduction was injected into 95 μL of potassium phosphate buffer solution (pH 7.4), and the resulting suspensions were incubated at 37° C. At designated times the suspensions were centrifuged. The supernatant was collected, and the precipitates were dissolved in acetonitrile. Both fractions were analysed by HPLC.

FIG. 1 shows HPLC analysis of the aniline compound before and after incubation. Fragmentation was only observed when the nitro group had been reduced, indicating that the increase in electron density provided by the nitrogen lone pair, induces fragmentation.

### Example 4

### Purified Reductase Assay

The bioreductive compound 18 (Example 1) was incubated with purified human cytochrome p450 reductase in hypoxic and normoxic conditions.

Bactosomal human NADPH-CYP reductase (Cypex, 12.7 mg/mL, 13900 nmol/min/mL) was used in combination with an NADPH-regenerating system (BD Biosciences), and the assay was carried out according to the manufacturer's protocol (BD Biosciences application note 467) at a compound 18 concentration of 250 nM. For hypoxic conditions, vials were deoxygenated by bubbling nitrogen prior to P450 addition and then transferred into a Bactron II (Shell laboratories). Samples were taken at different time points and analyzed by HPLC.

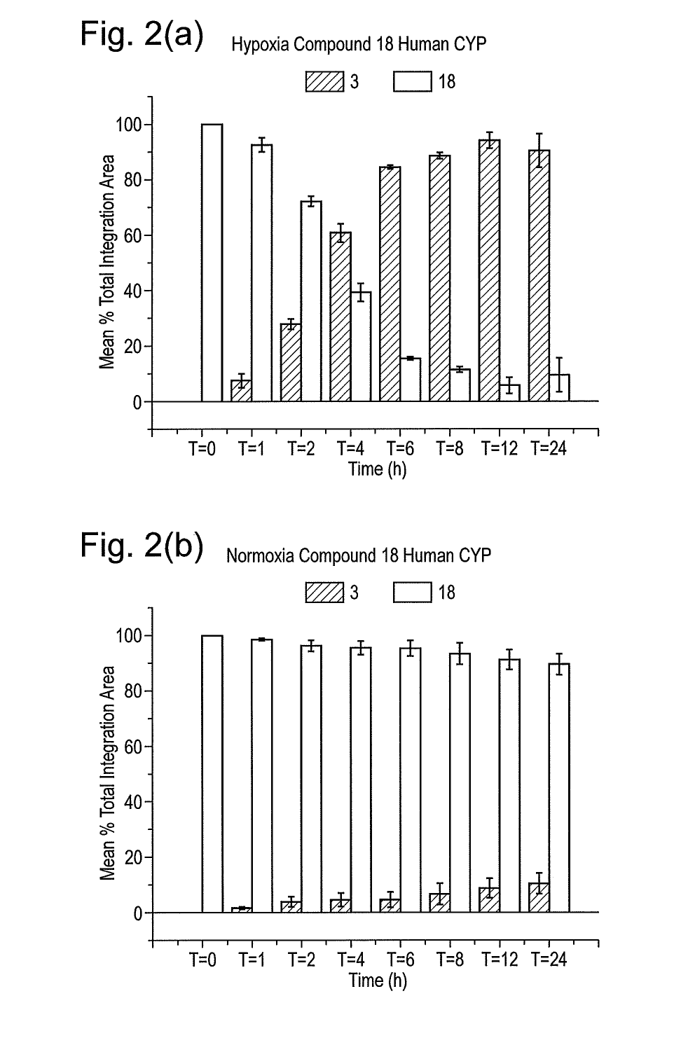

In hypoxic conditions, compound 18 undergoes reduction by human CYP, including fragmentation to release compound 3 via compound 19 (FIG. 2a). In contrast, in normoxic conditions, no activation was observed (FIG. 2b).

### Example 5

### Cell-Based Assays

Compound 21 was administered to cells in culture and incubated in (a) hypoxic and (b) normoxic conditions.

RKO (colorectal) cancer cell lines were cultured in DMEM medium containing 10% FBS, penicillin (100 U/mL) and streptomycin (100 μg/mL). RKO cell line was originally obtained from the ATCC and routinely mycoplasma tested and found to be negative. Experiments were carried out with cells at 75% confluence.

Hypoxia Treatments were carried out in a Bactron II (Shell laboratories), In vivo 400 (Ruskinn), or Heracell mixed gas incubator (Fisher Scientific) depending on the level of hypoxia required.

HPLC Analysis HPLC (Waters 2695 system) comprised an RPB column (100 mm×3.2 mm, 35° C.). Separation was achieved at a flow rate of 0.5 mL/min with a gradient of 30-95% acetonitrile in 10 mM formic acid over 6 min. Detection used a photodiode array spectrophotometer (Waters 2996), a mass spectrometer (Waters Micromass ZQ mass spectrometer), and a UV spectrometer (Waters 296 diode array detector) scanning from 210 nm to 400 nm with a resolution set at 3.6 nm. Injections of 10 μL were made.

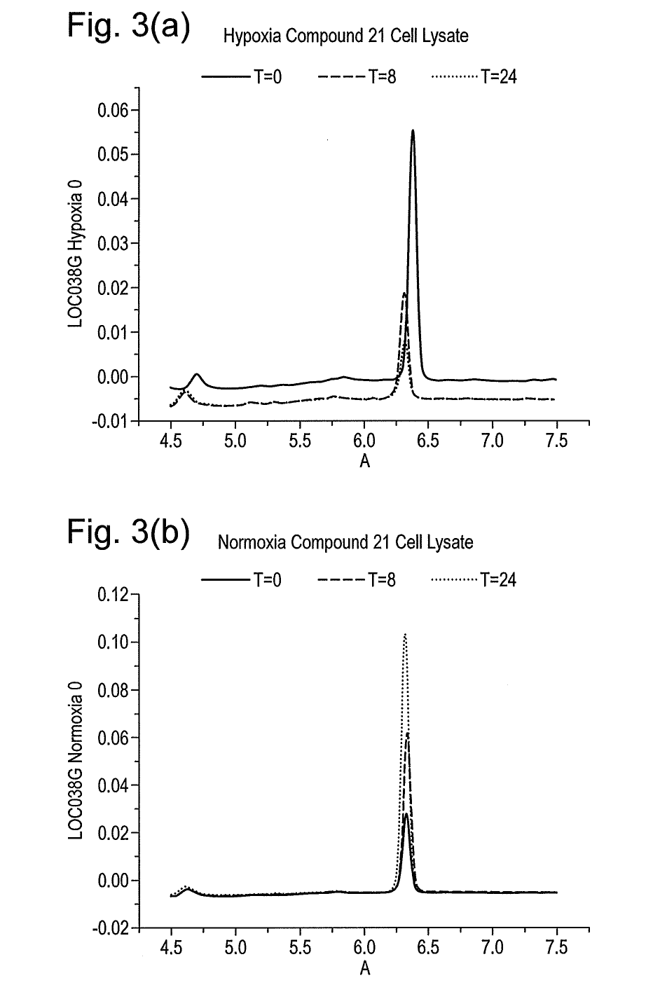

HPLC analysis of the lysate of cells treated with compound 1 is presented in FIGS. 3(a) (under hypoxia) and 3(b) (normoxia) and showed a hypoxia-dependent decrease in UV absorbance of the peak corresponding to compound 21 under hypoxia. Under normoxia, the UV absorbance peak of compound 21 continued to increase over a 24 h time period. This showed a hypoxia dependent activation of the biologically active compound paracetamol in cells.

### Example 6

### Fluorogenic Properties

The active fluorophore 22 corresponding to fluorogenic compound 18 was produced in order to study the fluorogenic properties of the compounds:

### (6-Aminoquinolin-5-yl)methanol (22)

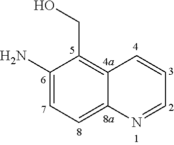 [SMILES: Nc1ccc2ncccc2c1CO]

To a solution of 8 (53 mg, 0.26 mmol) in DMF (1 mL), aq. NH₄Cl (0.25 mL, 10% w/v) and Zn powder (506 mg, 7.7 mmol, 30 eq) was added. The reaction mixture was stirred at ambient rt for 2 h, H₂O (10 mL) added, and the product extracted with EtOAc (3×10 mL). The combined organic layers were washed with distilled H₂O (3×10 mL), brine (2×10 mL), dried over MgSO₄, and concentrated in vacuo. The resulting residue was adsorbed onto Celite® and purified by silica gel chromatography, eluting with EtOAc to yield 22 (39 mg, 0.22 mmol, 85%) as a hygroscopic yellow solid: R_{f}: 0.31 (EtOAc).

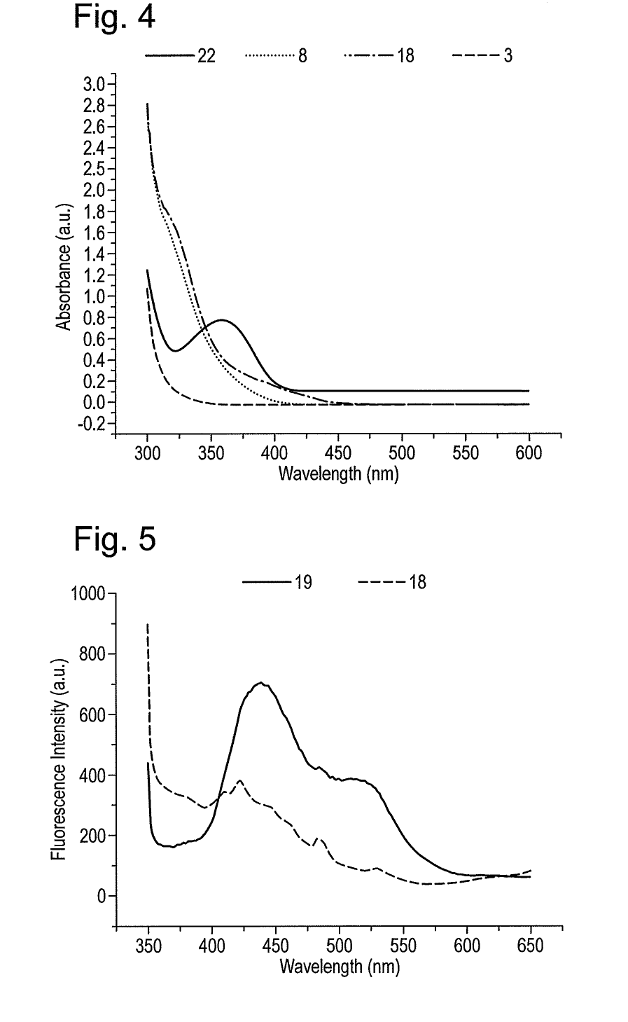

UV Vis-spectra of compound 18 (Example 1) as well as compounds 3, 8, and 22 at 0.5 mM in KPO₄ buffer solution were recorded. Only the amino quinoline 22 shows absorbance at 350 nm (See FIG. 4). Importantly, Example 1 itself was not fluorescent. Fluorescence emission spectra of compounds 18 and 19 are shown in FIG. 5. Compound 19 corresponds to compound 18 (Example 1), but has the nitro group replaced with an amine group.

### Example 7

### (E)-2-(2-(4-azidostyryl)-4H-chromen-4-ylidene)malononitrile (Compound A)

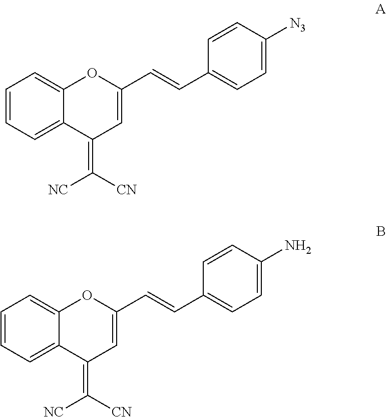 [SMILES: N#CC(C#N)=C1C=C(/C=C/c2ccc(N=[N+]=[N-])cc2)Oc2ccccc21 ; N#CC(C#N)=C1C=C(/C=C/c2ccc(N)cc2)Oc2ccccc21]

Compound A (Example 7) was synthesised in accordance with Sun et at (Chem. Commun., 2013, 49, 3890-3892). Fluorescence spectra of the compound, as well as its reduced counterpart compound B, was confirmed by fluorimetry.

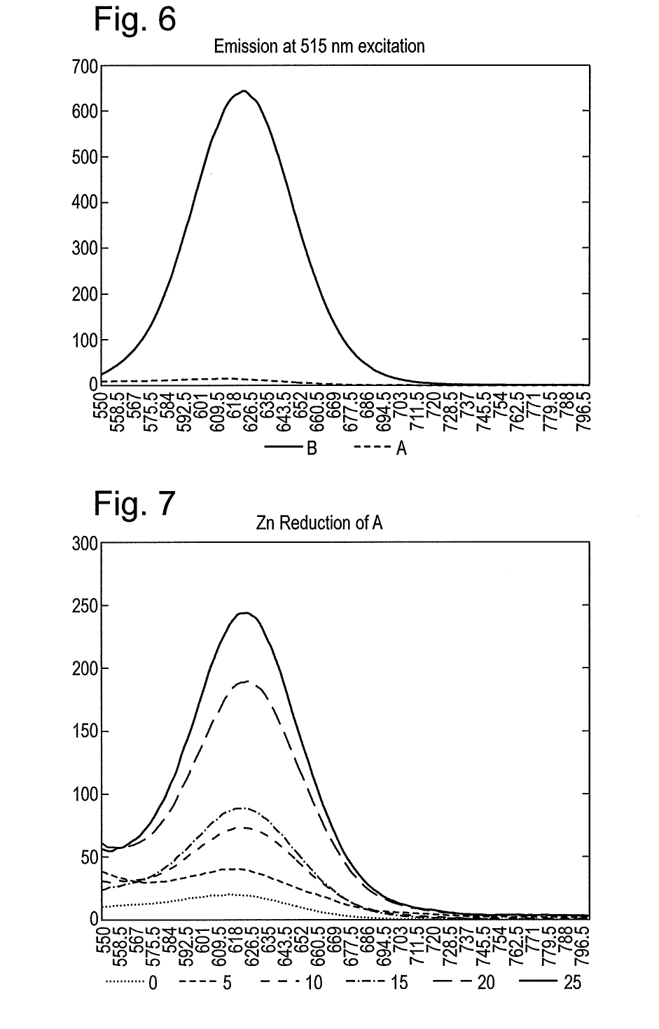

50 μM solutions of A and B were prepared in iPrOH and fluorimetry carried out using a Perkin Elmer Luminescence Spectrometer LS 50 B with an excitation wavelength of 515 nm. Results are depicted in FIG. 6. Approximately a 650 fold difference was observed in fluorescence emission at 515 nm.

### Example 8

### Zinc Mediated Reduction

Compound A was exposed to a zinc mediated reduction, and the increase in fluorescence monitored in a fluorimeter. To a solution of A (50 μM) in iPrOH (900 μL), 1% w/v NH4Cl (100 μL) and zinc (3 mg) was added. Fluorescence of the reaction mixture was monitored at 0, 5, 10, 15, 20 and 25 minutes after initiating reaction using a Perkin Elmer Luminescence Spectrometer LS 50 B with an excitation wavelength of 515 nm. The cuvette was shaken between each spectra run. The observed increase in fluorescence is depicted in FIG. 7.

### Example 9

### Cytochrome P450 Assay

Compound A was subject to a Cytochrome P450 reductase assay in accordance with the procedure of Cazares-Korner (CH-01 is a hypoxia-activated prodrug that sensitizes cells to hypoxia/reoxygenation through inhibition of Chk1 and Aurora A., ACS Chem. Biol. 2013 DOI:10.1021/cb4001537). This enzyme has been shown to activate bioreductive compounds with nitro functionalities under anaerobic conditions.

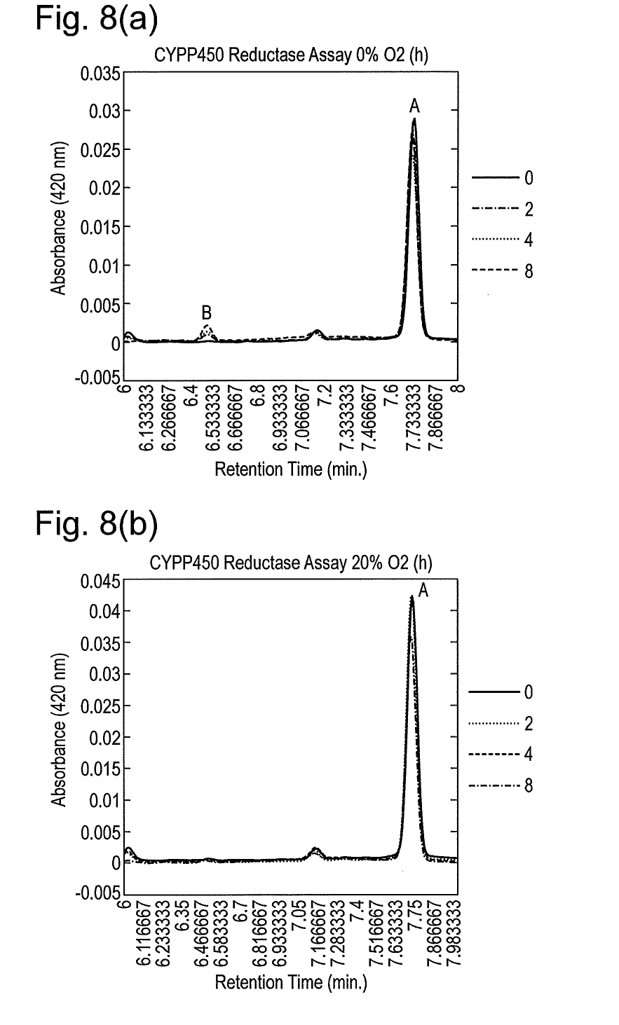

Under conditions susceptible to nitro reduction, compound A showed trace reductive conversion to compound B in this assay over 8 h under anaerobic conditions, as shown in FIG. 8. FIG. 8 records absorbance at 0, 2, 4 and 8 hours under hypoxic (FIG. 8a) and normoxic (FIG. 8b) conditions. Hypoxic and normoxic conditions were achieved in the same way as Example 4.

### Example 10

### Liver Microsomes

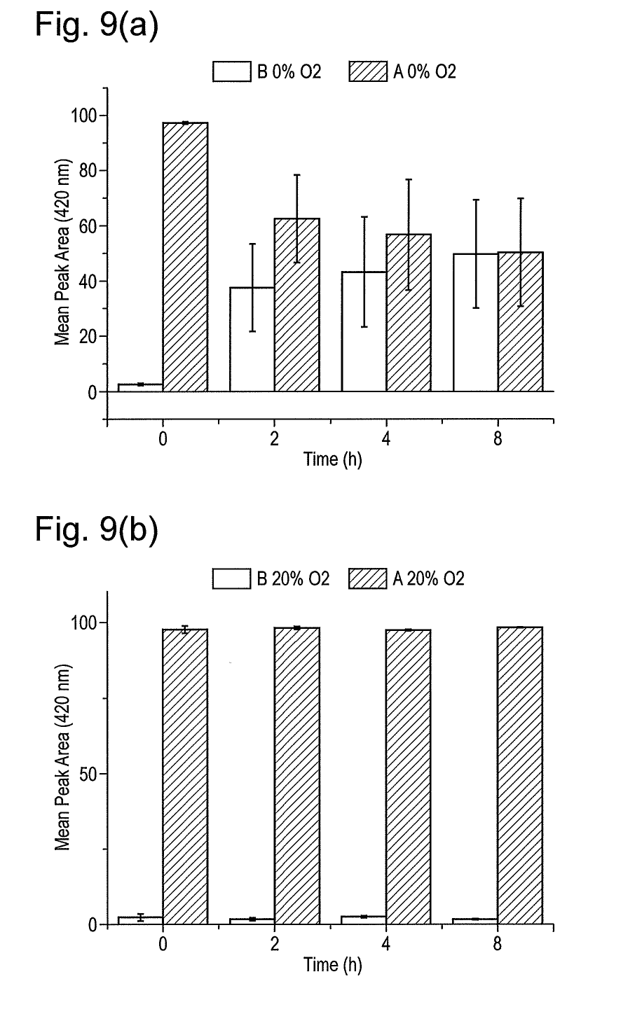

Compound A was exposed to human liver microsomes as supplied by Life Technologies (GIBCO® 2014). A solution of 26 mM NADPH, 66 mM MgCl₂ and 66 mM Glucose-6-Phosphate was prepared. To this solution was added 5 μL G6PDH((40 units/mL Glucose-6-Phosphate Dehydrogenase), 15 μL human liver microsomes (20 mg/mL), 455 μL 0.1 M KPO₄ buffer and 0.5 μL 10 mM compound A. Aliquots of 50 μL were taken at time points of 0 h, 2 h, 4 h and 8 h and quenched with 50 μL MeCN. The assay was carried out under both hypoxic and normoxic conditions and results are depicted in FIGS. 9a (hypoxic) 9b (normoxic).

### Example 11

### Purified Cytochrome P450 Enzymes

Compound A was subject to a cytochrome P450 assay using the following purified enzymes available from Cypex (CYP051 2014):

CYP1A2R (Human CYP1A2 and human CYP-reductase coexpressed in Escherichia coli);
CYP3A4R (Human CYP3A4 and human CYP-reductase coexpressed in Escherichia coli);
CYP2D6 (Human CYP2D6 expressed in Escherichia coli);
CYP2C19 (Human CYP2C19 expressed in Escherichia coli);
CYP2C9HR (Human CYP2C9 and human CYP-reductase coexpressed in Escherichia coli).

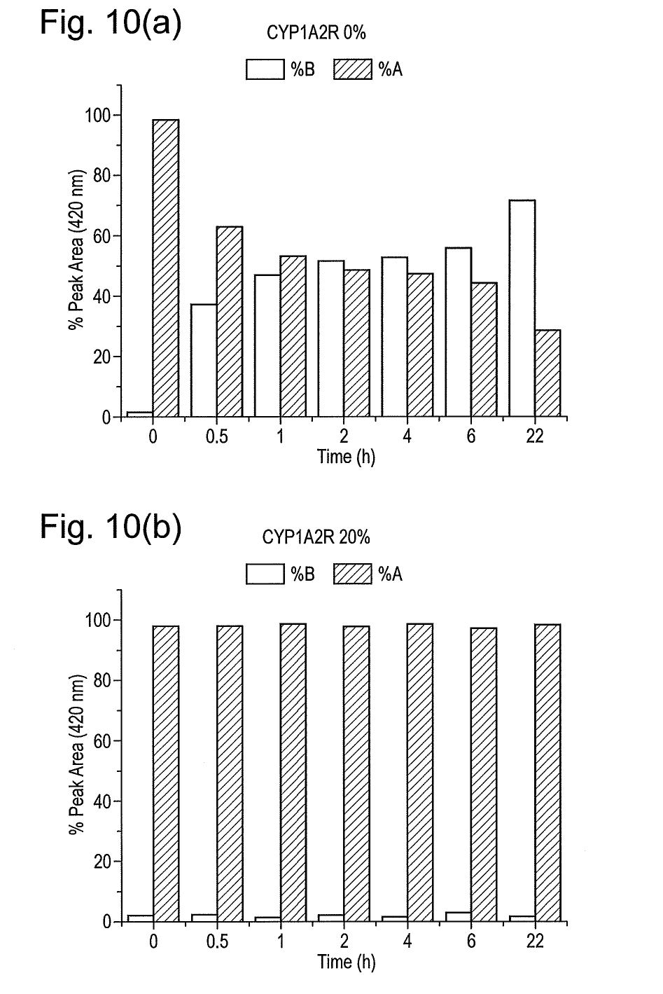

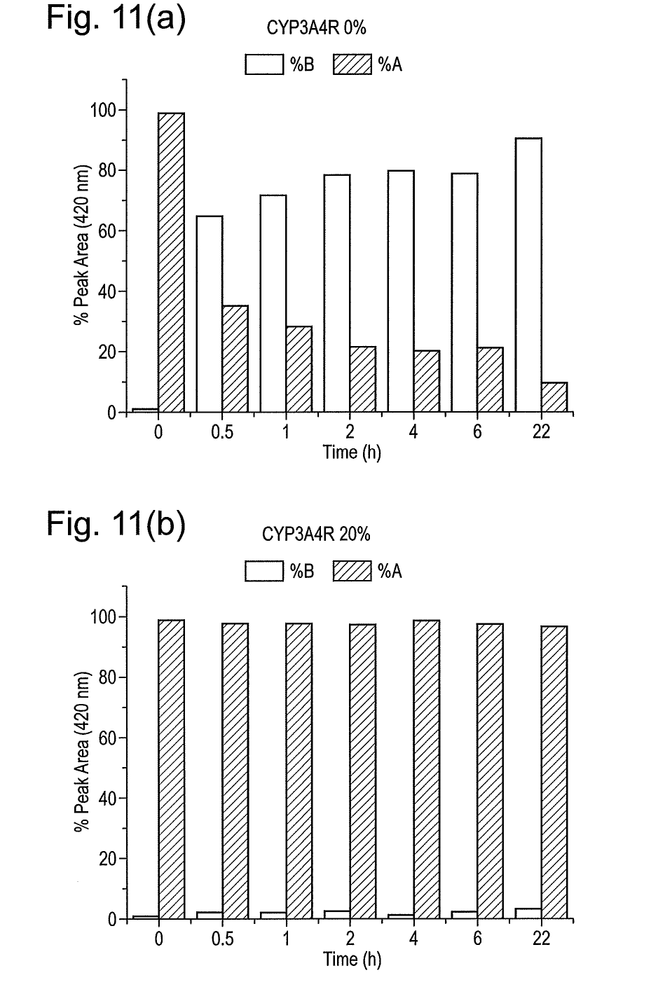

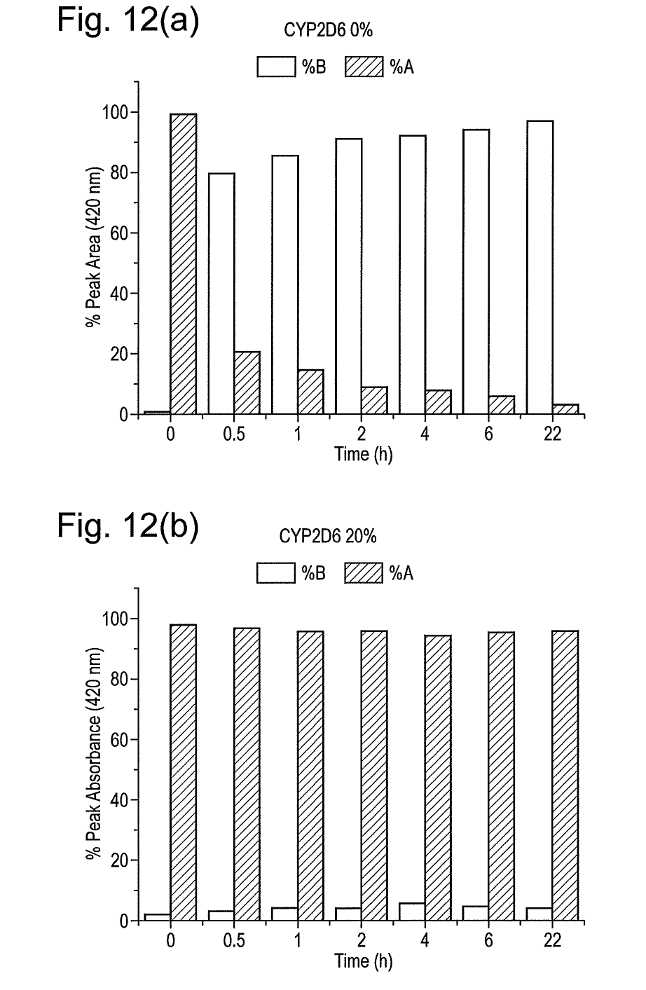

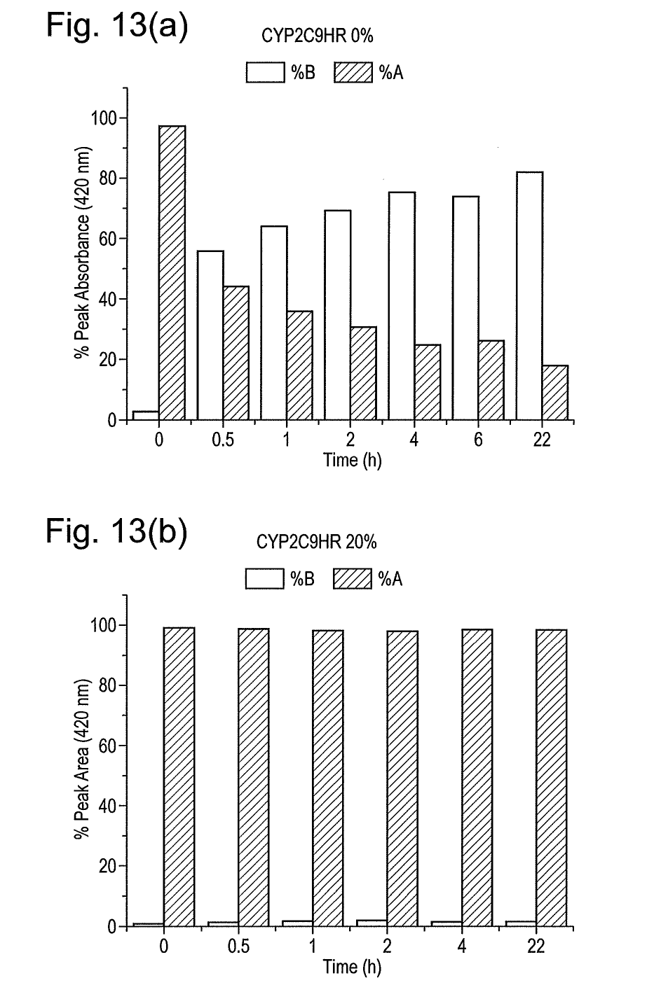

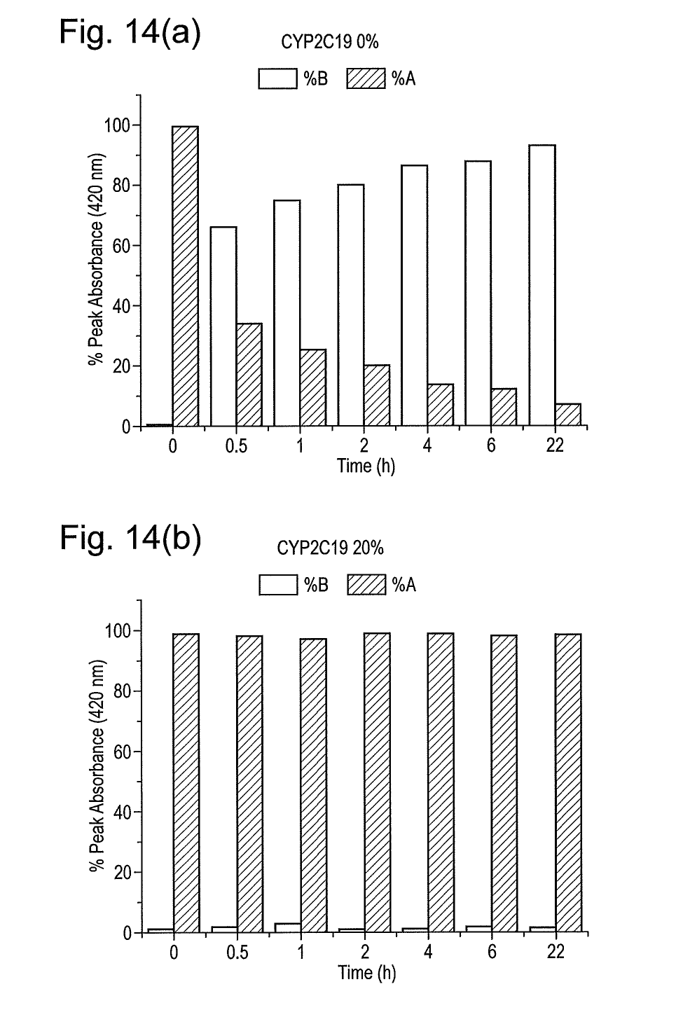

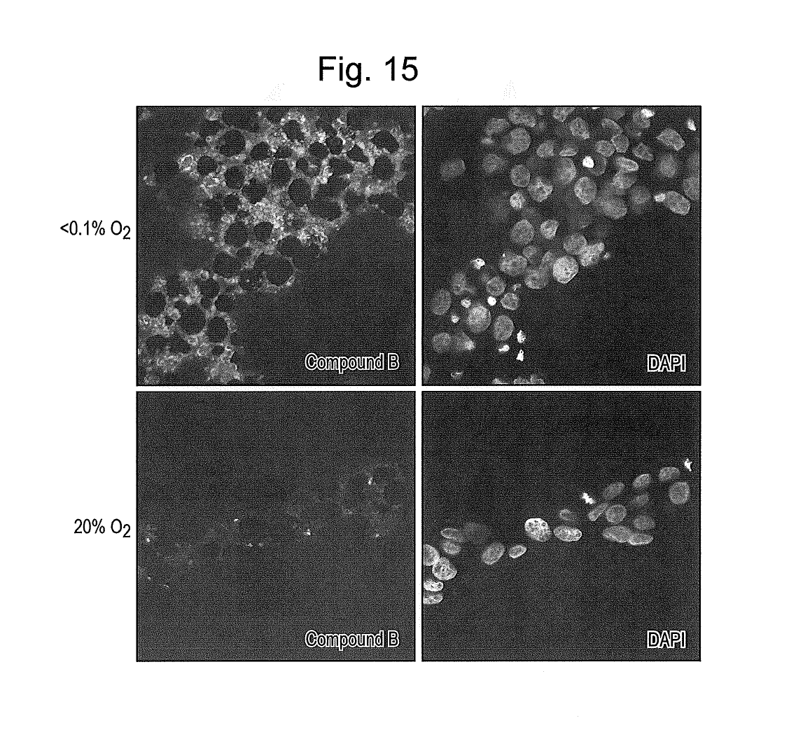

50 μL aliquots were taken and quenched with 50 μL MeCN. The resulting solution was then centrifuged at 17000 g. The resulting supernatant was removed from the enzyme pellet and analysed by HPLC. Results are set out in Tables 1 and 2 below and in FIGS. 10a and 10b to 14a and 14b.

TABLE 1

| Reagent | Volume | Final Concentration |
|---|---|---|
| 0.5M KPO4 | 200 μL | 100 mM |
| Solution A (26 mM NADPH, 66 mM | 50 μL | 1.3 mM NADPH, |
| MgCl2, 66 mM Glucose-6-Phosphate) | | 3.3 mM MgCl2, |
| | | 3.3 mM G6P |
| Solution B (40 units/mL Glucose-6- | 10 μL | 0.4 units/mL G6PDH |
| Phosphate Dehydrogenase) | | |
| H2O | 730 μL | |
| Substrate (1 mM DMSO) | 1 μL | 1 μM |
| P450 Reducatase 10.3 nmol/mL | 9 μL | 91.7 pmol/mL |

TABLE 2

| | Volume in 1 mL | |
|---|---|---|
| Enzyme (Supplier Concentration) | reaction volume | Volume H2O |
| CYP1A2R (5.4 nmol/mL) | 17 μL | 723 μL |
| CYP3A4R (6.1 nmol/mL) | 15 μL | 725 μL |
| CYP2D6 (3.3 nmol/mL) | 29 μL | 711 μL |
| CYP2C19 (4.2 nmol/mL) | 22 μL | 718 μL |
| CYP2C9HR (2.8 nmol/mL) | 33 μL | 707 μL |

### Example 12

### Cell Microscopy

HepG2 cells were treated with either compound A or compound B for 8 h as follows. 1 millions cells/6 cm dish with cover slips were seeded in DMEM media and left to adhere over 15 h. The media was replaced with fresh DMEM containing 1 μM of either compound A or compound B, and incubated at (a) <0/1% or (b) 20% O₂ for 8 h. After this time, the media was removed, the cells washed with PBS ×2 and fixed using 4% PFA for 15 min. The PFA was removed, the cells washed with PBS ×2 and the cover slides rinsed with water before mounting onto slides. Life Technologies Prolong® Gold Anti-Fade Reagent with DAPI was used as mounting media. The slides were left to dry overnight before analysis using a Zeiss Confocal Microscope. Results are depicted in FIG. 15.

### Example 13

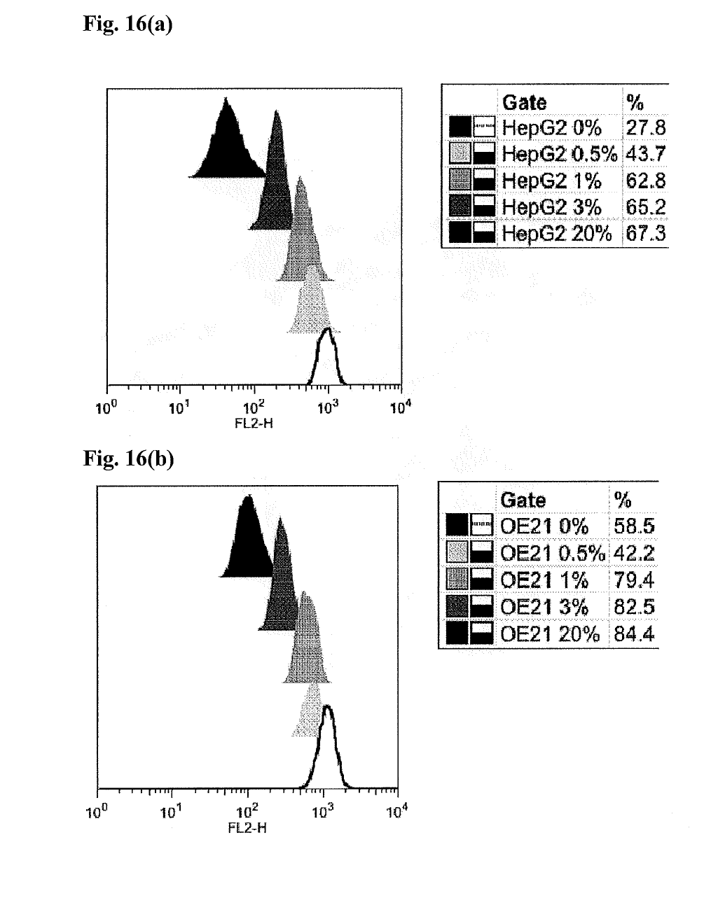

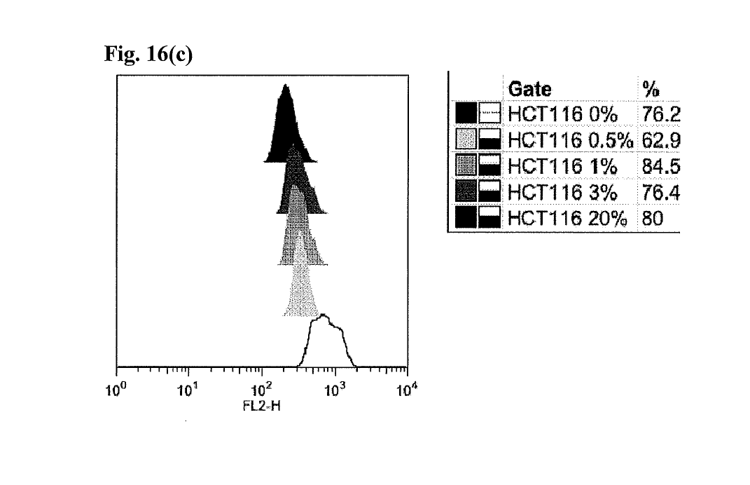

HepG2/OE21/HCT116 cells were seeded in 6 cm glass dishes at 0.5 million cells/dish and left to adhere over 16 h. Cells were then treated with 1 μM of compound A and incubated at the indicated O₂ concentration for 16 h. The media was removed, the cells washed with PBS three times, and trypsinised. The trypsin was removed at 2.5 g by centrifugation, and the resulting cell pellet resuspended in PBS for use in FACS analysis. FACS analysis was carried out using a Beckton Dickinson FACScan, and the resulting data analysed using FlowJo software. Results are depicted in FIGS. 16a (HepG2), 16b (OE21) and 16c (HCT116).

1. A fluorogenic compound comprising a biologically active component and a fluorogenic component, wherein the biologically active component is bound to the fluorogenic component at an active binding position, and wherein the fluorogenic component is cleavable from the biologically active component under hypoxic conditions to release a fluorescent compound and a biologically active compound.

2. A fluorogenic compound according to claim 1, wherein the fluorogenic component is of formula: 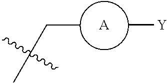
wherein the wavy line indicates the bond to the biologically active component; Y represents —NO₂ or —N₃; A comprises a phenyl, furyl, thienyl or imidazolyl moiety; and the group Y and the bond to the biologically active component are linked to the phenyl, furyl, thienyl or imidazolyl moiety on adjacent carbon atoms, or on carbon atoms which are separated by an odd number of bonds around the ring.

3. A fluorescent compound according to claim 2, wherein the group -A-Y is non-fluorescent and the group -A-NH₂ is fluorescent.

4. A fluorogenic compound according to claim 2 or 3, wherein A is a phenyl, furyl, thienyl or imidazolyl group which is fused or conjugated to one or more further aryl or heterocyclyl moieties.

5. A fluorogenic compound according to any one of claims 2 to 4, wherein the fluorescent compound which is released is a group HO—CH₂-A-NH₂.

6. A fluorogenic compound according to any one of claims 2 to 5, wherein A is an isoquinoline group.

7. A fluorogenic compound according to any one of the preceding claims, wherein the fluorogenic component is of formula: 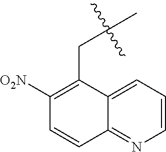 [SMILES: *Cc1c([N+](=O)[O-])ccc2ncccc12]
wherein the wavy line indicates the bond to the biologically active component.

8. A fluorogenic compound according to any one of the preceding claims, wherein the fluorogenic component is of formula: 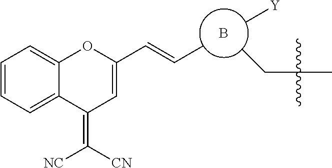
wherein the wavy line indicates the bond to the biologically active component; Y is —NO₂ or —N₃; and B is a phenyl, furyl, thienyl or imidazolyl ring wherein the group Y and the bond to the biologically active component are linked to B on adjacent carbon atoms, or on carbon atoms which are separated by an odd number of bonds around the ring.

9. A fluorogenic compound according to any one of the preceding claims, wherein the fluorogenic component is of formula: 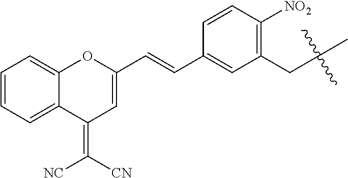 [SMILES: *Cc1cc(/C=C/C2=CC(=C(C#N)C#N)c3ccccc3O2)ccc1[N+](=O)[O-]]
or

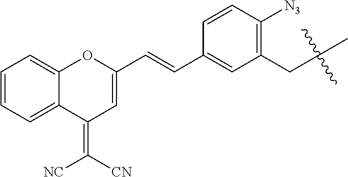 [SMILES: *Cc1cc(/C=C/C2=CC(=C(C#N)C#N)c3ccccc3O2)ccc1N=[N+]=[N-]]

10. A pharmaceutical composition comprising a fluorogenic compound according to any one of the preceding claims and a pharmaceutically acceptable carrier or diluent.

11. A fluorogenic compound according to any one of claims 1 to 9, or a composition according to claim 10, for use in a method of treatment of a hypoxia-related disease or disorder in a human or animal subject.

12. A fluorogenic compound or composition for use according to claim 11, wherein the hypoxia-related disease or disorder is tumour.

13. A fluorogenic compound or composition for use according to claim 11, wherein the hypoxia-related disease or disorder is a disease or disorder of a hypoxic organ.

14. A fluorogenic compound or composition for use according to claim 13, wherein the hypoxic organ is the kidney and the compound is for use in treating a kidney disease or disorder.

15. A fluorogenic compound or composition for use according to any one of claims 11 to 14, wherein the method further comprises carrying out fluorescence imaging on the subject.

16. A method of treatment of a hypoxia-related disease or disorder in a human or animal subject comprising administering to the subject an effective amount of a fluorogenic compound according to any one of claims 1 to 9 or a composition according to claim 10.

17. A method according to claim 16, wherein the hypoxia-related disease or disorder is tumour.

18. A method according to claim 16, which is a method for the treatment of a disease or disorder of a hypoxic organ.

19. A method according to claim 18, wherein the hypoxic organ is the kidney and the method is for the treatment of a kidney disease or disorder.

20. A method according to any one of claims 16 to 19, further comprising carrying out fluorescence imaging on the subject.
